SEQUENCE LISTING (7 sequences)

SEQ ID NO: 1 - DNA, 20 bp, Artificial
  Forward primer for PCR of beta-actin
acaaaacctaacttgcgcag

SEQ ID NO: 2 - DNA, 20 bp, artificial
  Reverse primer for PCR of beta-actin
tcctgtaacaacgcatctca

SEQ ID NO: 3 - DNA, 23 bp, artificial
  Forward primer for PCR of tyrosinase
tcttctcctcttggcagattgtc

SEQ ID NO: 4 - DNA, 22 bp, artificial
  Reverse primer for PCR of tyrosinase
tgtcatggtttccaggattacg

SEQ ID NO: 5 - DNA, 765 bp, Saponaria officinalis
  CDS (1)..(765)
atggtcacatcaatcacattagatctagtaaatccgaccgcgggtcaatactcatcttttgtggataaaatccgaaacaacgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcagatcagaaattacttccgccgagtcaaccgcccttttcccagaggccacaactgcaaatcagaaagctttagaatacacagaagattatcagtcgattgaaaagaatgcccagataacacaaggagatcaaagtagaaaagaactcgggttggggattgacttactttcaacgtccatggaagcagtgaacaagaaggcacgtgtggttaaagacgaagctagattccttcttatcgctattcagatgacggctgaggcagcgcgatttaggtacatacaaaacttggtaatcaagaactttcccaacaagttcaactcggaaaacaaagtgattcagtttgaggttaactggaaaaaaatttctacggcaatatacggggatgccaaaaacggcgtgtttaataaagattatgatttcgggtttggaaaagttaggcaggtgaaggacttgcaaatgggactccttatgtatttgggcaaaccaaagtga

SEQ ID NO: 6 - protein, 254 aa, Saponaria officinalis
MVTSITLDLVNPTAGQYSSFVDKIRNNVKDPNLKYGGTDIAVIGPPSKEKFLRINFQSSRGTVSLGLKRDNLYVVAYLAMDNTNVNRAYYFRSEITSAESTALFPEATTANQKALEYTEDYQSIEKNAQITQGDQSRKELGLGIDLLSTSMEAVNKKARVVKDEARFLLIAIQMTAEAARFRYIQNLVIKNFPNKFNSENKVIQFEVNWKKISTAIYGDAKNGVFNKDYDFGFGKVRQVKDLQMGLLMYLGKPK

SEQ ID NO: 7 - DNA, 792 bp, Artificial
  Saporin-6 codon optimized for expression in humans
gagctcgccaccatggtgaccagcatcaccctggacctggtgaaccccacagccggccagtacagcagcttcgtggacaagatccggaacaacgtgaaggaccccaacctgaagtacggcggcaccgatatcgccgtgatcggccctcccagcaaagagaagttcctgcggatcaacttccagagcagccggggcacagtgtccctgggcctgaagcgggacaacctgtacgtggtggcctacctggccatggacaacaccaacgtgaacagagcctactacttcagaagcgagatcaccagcgccgagagcaccgccctgtttccagaggccaccaccgccaaccagaaggccctggaatacaccgaggactaccagagcatcgagaagaacgcccagatcacccagggcgaccagagcagaaaagagctgggcctgggcatcgacctgctgtccacctccatggaagccgtgaacaagaaagcccgggtggtgaaggacgaggcccggttcctgctgatcgccatccagatgaccgccgaggccgccagattccggtacatccagaacctggtgatcaagaacttccccaacaagttcaacagcgagaacaaagtgatccagttcgaagtgaactggaagaagatcagcaccgccatctacggcgacgccaagaacggcgtgttcaacaaggactacgacttcggcttcggcaaagtgcgccaggtgaaggacctccagatgggcctgctgatgtacctgggcaagcccaagtgatgactgcagggtacc

(19) United States
(12) Patent Application Publication
(10) Pub. No.: US 2010/0297091 A1
(43) Pub. Date: Nov. 25, 2010

(54) COMPOSITIONS AND METHODS FOR TREATMENT OF MELANOMAS

(76) Inventors: James J-L WANG, Monterey Park, CA (US); Mike CHEN, Duarte, CA (US)

Correspondence Address: PERKINS COIE LLP, POST OFFICE BOX 1208, SEATTLE, WA, 98111-1208, US

(21) Appl. No.: 12/780,831

(22) Filed: May 14, 2010

Related U.S. Application Data
(60) Provisional application No. 61/178,911, filed on May 15, 2009.

(51) Int. Cl. A61K 35/12; A61N 7/00; C07H 21/04; C12N 15/63; C12N 15/85; C12N 15/88; C12N 15/861; C12N 5/10; A61K 31/7088; A61P 35/00

(52) U.S. Cl. 424/93.21; 604/22; 536/23.1; 536/23.2; 435/320.1; 435/325; 514/44 R

(57) ABSTRACT

In certain embodiments, an expression construct is provided that contains a tissue-specific promoter such as tyrosinase linked to one or more cytotoxin genes. The cytotoxin genes may be saporin genes. In certain embodiments, a vector is provided that contains an expression construct as discussed above. The vector may be an adeno-associated virus or an adenovirus. Further, neural stem cells may be provided that contain and/or produce a vector, which in turn contains an expression construct containing a tissue-specific promoter linked to one or more cytotoxin genes. In certain embodiments, methods of using the neural stem cells provided herein for production and delivery of an animal virus vector are provided. Methods for treating melanomas in a subject in need thereof are also provided by delivering an expression construct that contains a tissue-specific promoter linked to one or more cytotoxins. In certain embodiments, the melanoma is metastatic, and in certain embodiments the melanoma is a brain melanoma.

### RELATED APPLICATION

This application claims priority to U.S. Provisional Patent Application No. 61/178,911, filed May 15, 2009, the content of which is incorporated by reference herein in its entirety.

### BACKGROUND

Melanoma, particularly metastatic melanoma, is associated with a very poor prognosis. Conventional therapies such as surgery, radiation, and chemotherapy are largely ineffective. Median survival in patients with stage 1V disease is 6-10 months (Liu 2007). In addition, among primary tumors, melanomas have one of the highest propensities to metastasize to the brain. Brain metastasis is very resistant to treatments, and accounts for approximately 20-54% of deaths in patients with melanoma. Therefore, there is a need in the art for new compositions and methods for the treatment of melanomas.

### SUMMARY

The present application discloses a novel tissue-specific promoter gene therapy approach for treating melanoma.

In certain embodiments, an expression construct is provided that contains a tissue-specific promoter linked to one or more cytotoxin genes. In certain embodiments, the tissue-specific promoter is a tyrosinase promoter. In certain embodiments, the construct also includes one or more enhancer elements. In certain embodiments, one or more of the cytotoxin genes are cytotoxic to both quiescent and rapidly dividing cells. In certain of these embodiments, one or more of the cytotoxin genes encode ribonucleotide inactivating proteins, and in certain of these embodiments one or more of the cytotoxin genes are saporin genes.

In certain embodiments, a vector is provided that contains an expression construct containing a tissue-specific promoter linked to one or more cytotoxin genes. In certain embodiments, the tissue-specific promoter is a tyrosinase promoter. In certain embodiments, the construct also includes one or more enhancer elements. In certain embodiments, one or more of the cytotoxin genes are cytotoxic to both quiescent and rapidly dividing cells. In certain of these embodiments, one or more of the cytotoxin genes encode ribonucleotide inactivating proteins, and in certain of these embodiments one or more of the cytotoxin genes are saporin genes. In certain embodiments, the vector is an animal virus vector or a hybrid animal virus vector, and in certain of these embodiments the vector is an adeno-associated virus or an adenovirus.

In certain embodiments, neural stem cells are provided that contain and/or produce a vector which in turn contains an expression construct containing a tissue-specific promoter linked to one or more cytotoxin genes. In certain embodiments, the tissue-specific promoter is a tyrosinase promoter. In certain embodiments, the construct also includes one or more enhancer elements. In certain embodiments, one or more of the cytotoxin genes encode ribonucleotide inactivating proteins, and in certain of these embodiments one or more of the cytotoxin genes are saporin genes. In certain embodiments, the vector is an animal virus vector or a hybrid animal virus vector, and in certain of these embodiments the vector is an adeno-associated virus or an adenovirus. Provided herein in certain embodiments are methods of using the neural stem cells provided herein for production and delivery of an animal virus vector.

In certain embodiments, methods are provided for treating melanomas in a subject in need thereof by delivering an expression construct that contains a tissue-specific promoter linked to one or more cytotoxins. In certain embodiments, the expression construct is delivered via a vector as provided herein, and in certain of these embodiments the vector is delivered via a neural stem cell as provided herein. In certain embodiments, the melanoma is metastatic, and in certain embodiments the melanoma is a brain melanoma.

In certain embodiments, adeno-associated virus constructs that express a cytotoxin (AAV-cytotoxin) and methods of making such constructs are provided. In certain embodiments, these AAV-cytotoxin constructs have the following properties: the expression of the cytotoxin is under the control of a tissue specific promoter and the activity of the tissue specific promoter is under the control of a tetracycline inducible system.

### BRIEF DESCRIPTION OF THE DRAWINGS

FIG. 1: In vitro activity of human enhancer (hE)-tyrosinase promoter (TyrP) construct. The basis of selective destruction of melanoma cells is that melanoma cells possess a cellular milieu in which TyrP is active whereas most other cells do not. A. Constructs were inserted into pBlue vector, a promoter activity reporter. B. Activity of TyrP linked to either one (1 hE) or two (2hE) copies of the human enhancer was assayed by transiently transfecting melanoma (HTB72) or nonmelanoma (U251) cell lines and measuring beta-galactosidase activity. Positive control cells were transfected with pBlue containing reporter gene linked to the constitutively active cytomegalovirus (CMV) promoter. Untransfected cell lysates served as negative controls. Results shown are the mean of three individual experiments. Bars represent standard error of the mean (SEM). Promoter activity was significantly higher (P<0.01) in HTB72 melanoma cells than in nonmelanoma cells or negative controls.

FIG. 2: Cytotoxicity of saporin (SA) cytotoxin in melanoma and nonmelanoma cells. Saporin cytotoxicity was confirmed by linking the SA gene to a constitutively active promoter. A. The SA gene was inserted into pIRES-enhanced green fluorescent protein (EGFP) under control of the CMV promoter. B. SA cytotoxicity was assayed by transiently transfecting HTB72 melanoma and U251 nonmelanoma cells with SA-CMV or CMV-EGFP control and measuring cell killing. Cytotoxicity was measured in relative light units (RLUs). Results shown are the mean of three individual experiments. Bars represent SEM. Cytotoxicity was significantly increased (P<0.01) in cells transfected with SA versus cells transfected with control vector.

FIG. 3: Cytotoxicity of TyrP-SA in melanoma cells. The saporin gene was placed under the control of TyrP linked to one to four copies of hE (1TY, 2TY, 3TY, 4TY), and cytotoxicity was measured by transiently transfecting (A) HTB72 and (B) WYC1 melanoma cells with the resultant constructs. Negative control cells were transfected with a pcDNA control vector, and positive control cells were transfected with a CMV-SA vector. Cytotoxicity was measured in relative light units (RLUs). Results shown are the mean of three individual experiments. Bars represent SEM. Cytotoxicity was significantly increased (P<0.01) in all cells transfected with hE-TyrP-SA constructs versus cells transfected with the negative control vector. All hE-TyrP-SA constructs caused similar levels of cytotoxicity to the positive control vector.

FIG. 4: Non-cytotoxicity of TyrP-SA in nonmelanoma cells. The saporin gene was placed under the control of a TyrP promoter linked to two copies of hE, and cytotoxicity was measured by transiently transfecting U251 glioma cells with the resultant construct. Negative control cells were transfected with an empty control vector, and positive control cells were transfected with a CMV-SA vector. Cytotoxicity was measured in relative light units (RLUs). Results shown are the mean of three individual experiments. Bars represent SEM. Positive control vectors caused significant (P<0.05) cell death, but TyrP-SA did not cause significant cytotoxicity in these nonmelanoma cells.

FIG. 5: Comparison of CMV-optimized SA versus TyrP-optimized SA cytotoxicity in U251 glioma cells. An optimized saporin gene (SEQ ID NO:6) was placed under the control of CMV (“CMV-saporin6”) or 2hE-TyrP (“2hE-TyrP-saporin6”) and cytotoxicity was measured by transiently transfected U251 glioma cells with 1.4 μg of vector and assessing cell viability at 48 hours. Negative control cells were transfected with empty pIRES vector. Cytotoxicity was increased in cells transfected with CMV-optimized SA versus cells transfected with empty vector, but not in cells transfected with 2hE-TyrP-optimized saporin.

FIG. 6: Comparison of CMV-optimized SA versus TyrP-optimized SA cytotoxicity in U251 glioma cells. An optimized saporin gene (SEQ ID NO:6) was placed under the control of CMV (“CMV-saporin6”) or 2hE-TyrP (“2hE-TyrP-saporin6”) and cytotoxicity was measured by transiently transfected U251 glioma cells with 2.1 μg of vector and assessing cell viability at 48 hours. Negative control cells were transfected with empty pIRES vector. Cytotoxicity was significantly (P<0.01) increased in cells transfected with CMV-optimized SA versus cells transfected with empty vector, but not in cells transfected with 2hE-TyrP-optimized saporin.

FIG. 7: Comparison of CMV-optimized SA versus TyrP-optimized SA cytotoxicity in U251 glioma cells. An optimized saporin gene (SEQ ID NO:6) was placed under the control of CMV (“CMV-saporin6”) or 2hE-TyrP (“2hE-TyrP-saporin6”) and cytotoxicity was measured by transiently transfected U251 glioma cells with 0.7 μg of vector and assessing cell viability at 72 hours. Negative control cells were transfected with empty pIRES vector. Cytotoxicity was significantly (P<0.01) increased in cells transfected with CMV-optimized SA versus cells transfected with empty vector, but not in cells transfected with 2hE-TyrP-optimized saporin.

FIG. 8: Comparison of CMV-optimized SA versus TyrP-optimized SA cytotoxicity in HTB65 melanoma cells. An optimized saporin gene (SEQ ID NO:6) was placed under the control of CMV (“CMV-saporin6”) or 2hE-TyrP (“2hE-TyrP-saporin6”) and cytotoxicity was measured by transiently transfected HTB65 melanoma cells with 0.7 μg of vector and assessing cell viability at 96 hours. Negative control cells were transfected with empty pIRES vector. Cytotoxicity was increased in cells transfected with CMV-optimized SA versus cells transfected with empty vector, but not in cells transfected with 2hE-TyrP-optimized saporin.

FIG. 9: Comparison of CMV-optimized SA versus TyrP-optimized SA cytotoxicity in HTB65 melanoma cells. An optimized saporin gene (SEQ ID NO:6) was placed under the control of CMV (“CMV-saporin6”) or 2hE-TyrP (“2hE-TyrP-saporin6”) and cytotoxicity was measured by transiently transfected HTB65 melanoma cells with 1.4 μg of vector and assessing cell viability at 96 hours. Negative control cells were transfected with empty pIRES vector. Cytotoxicity was significantly (P<0.01) increased in cells transfected with CMV-optimized SA versus cells transfected with empty vector, but only slightly increased in cells transfected with 2hE-TyrP-optimized saporin.

FIG. 10: Comparison of CMV-optimized SA versus TyrP-optimized SA cytotoxicity in HTB65 melanoma cells. An optimized saporin gene (SEQ ID NO:6) was placed under the control of CMV (“CMV-saporin6”) or 2hE-TyrP (“2hE-TyrP-saporin6”) and cytotoxicity was measured by transiently transfected HTB65 melanoma cells with 2.1 μg of vector and assessing cell viability at 96 hours. Negative control cells were transfected with empty pIRES vector. Cytotoxicity was significantly (P<0.01) increased in cells transfected with CMV-optimized SA and in cells transfected with 2hE-TyrP-optimized saporin versus control cells transfected with empty vector.

FIG. 11: Schematic diagram of the inducible tissue-specific saporin expressing AAV (adeno-associated virus) construct. Expression of saporin is under the control of the melanocyte specific promoter and the tetracycline inducible system. ITR, Inverted terminal repeat; CMV, cytomegalovirus promoter; tTS, tetracycline-controlled transcriptional silencer; TRE, tetracycline response element; 2hE TyrP, tyrosinase promoter with two copies of human enhancers.

FIG. 12: mRNA expression profiles of tyrosinase in different human tissue and melanoma cell lines. Shown are quantitative RT-PCR results. The values are normalized with level of GAPDH. A trace amount of tyrosinase was detected in heart and its value was set to 1. Tyrosinase was also detected in adipose and brain tissues, but the expression level in brain was 10 million less than in melanoma cells.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

The following abbreviations are used herein: AAV, adeno-associated virus; HE, enhancer element; NSC, neuronal stem cell; RIP, ribosome inactivating protein; TRE, tetracycline response element; tTS, tetracycline-controlled transcriptional silencer; TyrP, tyrosinase promoter.

The phrase “therapeutically effective amount” as used herein refers to an amount of a compound that produces a desired therapeutic effect. The precise therapeutically effective amount is an amount of the composition that will yield the most effective results in terms of efficacy in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy (Gennaro ed. 20^{th} edition, Williams & Wilkins PA, USA) (2000).

“Treating” or “treatment” of a condition as used herein may refer to preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof. With regard to cancer, “treating” or “treatment” may refer to inhibiting or slowing neoplastic and/or malignant cell growth, proliferation, and/or metastasis, preventing or delaying the development of neoplastic and/or malignant cell growth, proliferation, and/or metastasis, or some combination thereof. With regard to a tumor, “treating” or “treatment” may refer to eradicating all or part of a tumor, inhibiting or slowing tumor growth and metastasis, preventing or delaying the development of a tumor, or some combination thereof.

Conventional therapies such as surgery, radiation, and chemotherapy have proven largely ineffective for treatment of metastatic melanomas. The ability to kill cancer cells in a targeted manner while minimizing damage to surrounding normal tissue presents a technical challenge. Many gene and viral therapy approaches have been attempted, but achieving successful targeted gene expression has been a major challenge. Therefore, there is a need for improved gene and viral therapy compositions and methods.

Provided herein are novel compositions and methods for treating melanoma, including melanoma that has metastasized. The experimental results set forth below describe the construction and characterization of an embodiment of the expression constructs provided herein, comprising TyrP, one or more enhancers, and the saporin cytotoxin gene. Tyrosinase is the rate-limiting enzyme in human melanin synthesis, and its expression is restricted to pigmented cells including melanoma cells (Siders 1996, Toyoda 2004). Since tyrosinase is expressed in melanoma cells but not in most normal tissues (e.g., brain tissue), utilization of the tyrosinase promoter allows for specific targeting of melanoma cells. Indeed, as shown in the experimental results, TyrP is active in melanoma cells but not nonmelanoma cells, allowing for targeted expression of a linked cytotoxin gene. Transfection of melanoma cells with a TyrP-saporin cytotoxin expression construct resulted in cell killing, whereas transfection of nonmelanoma cells did not. Similar results were obtained using either wild-type or optimized saporin genes in the construct. Therefore, insertion of the TyrP-cytotoxin expression constructs provided herein allow for the targeted killing of melanoma cells while leaving normal cells unharmed.

In certain embodiments, compositions are provided comprising an expression construct that comprises a tissue specific promoter optionally linked to one or more enhancers and one or more cytotoxin genes. In certain embodiments, the tissue specific promoter is TyrP.

In certain embodiments, the one or more enhancers comprise one or more enhancers that are normally linked to the tissue specific promoter.

In certain embodiments, one or more of the cytotoxin genes encode ribosome inactivating proteins (RIPs), which are potent inhibitors of protein synthesis that act in a cell cycle-independent manner (Zarovni 2007). The ability to act in a cell-cycle independent manner allows RIPs to induce cytotoxicity in both aggressively growing tumors and more slow growing tumors. RIPs extracted from certain plants remove an adenine residue from ribosomal RNA, preventing the 60s subunits of eukaryotic ribosomes from binding to elongation factor (Stirpe 2006). In certain embodiments, the RIPs employed in the compositions and methods disclosed herein may be either type I or type II. Type I RIPs that may be used in the compositions and methods disclosed herein may be, for example, saporin, particularly saporin-6.

Saporin is a single chain RIP extracted in large quantities from the seeds of Saponaria officinalis, and is the most cytotoxic of the type I RIPs studied to date (Benatti 1989, Benatti 1991, Bagga 2003, Sikriwal 2008). One advantage of using saporin is that it lacks an insertion domain and is only toxic in a cell when expressed (Stirpe 1992), making it particularly safe for administration in the methods disclosed herein.

In certain embodiments of the compositions and methods disclosed herein, one or more of the cytotoxin genes in the expression construct encode a native saporin-6 protein having the amino acid sequence set forth in SEQ ID NO:6. In certain of these embodiments, the cytotoxin gene has the native nucleotide sequence set forth in SEQ ID NO:5. In other of these embodiments, the cytotoxin gene comprises a nucleotide sequence that has been codon optimized for expression in humans, such as for example the nucleotide sequence set forth in SEQ ID NO:7. In these embodiments, the optimized gene may encode a saporin protein having the amino acid sequence in SEQ ID NO:6. In other embodiments, the saporin protein encoded by the optimized nucleotide sequence may vary slightly from the amino acid sequence of SEQ ID NO:6, while still possessing the activity that is the same as or similar to native saporin-6 protein.

Type II RIPs that may be used in the compositions and methods disclosed herein may be, for example, ricin, the most well studied of the type II RIPs (Stirpe 2006). In other embodiments, the cytotoxin genes may be selected from one or more toxic suicide genes such as herpes simplex thymidine kinase, small globular protein, or Bax.

In certain embodiments, compositions are provided that comprise a vector comprising one or more of the expression constructs provided herein. Vectors that may be utilized include, for example, animal viruses, hybrid animal viruses, liposomes, plasmids, phagemids, cosmids, artificial chromosomes such as yeast artificial chromosome (YAC), bacterial artificial chromosome (BAC), or P1-derived artificial chromosome (PAC), and bacteriophages such as lambda phage or M13 phage. Categories of animal viruses and hybrid animal viruses that may be used as vectors include adenovirus, adeno-associated virus, retrovirus (including lentivirus), herpesvirus (e.g., herpes simplex virus), poxvirus, baculovirus, papillomavirus, and papovavirus (e.g., SV40).

In certain embodiments, the vector is an adeno-associated virus (AAV). AAV has elicited much interest as a gene therapy vector due to its lack of pathogenicity and its ability to efficiently transduce both dividing and non-dividing cells. AAV is also attractive as a vector candidate because of its unique ability to preferentially integrate its genome into the AAVS1 site on human chromosome 19, a process mediated by the AAV-derived Rep68 and Rep78 proteins. In certain embodiments wherein the vector disclosed herein is an AAV vector, the AAV is replication competent and minimally immunogenic, and in certain of these embodiments the AAV is serotype 2, which has a natural affinity for melanoma cells (Hacker 2005). In other embodiments, the AAV is replication incompetent.

In certain embodiments, an AAV vector is used for expression of a cytotoxin gene, wherein the cytotoxin gene is under the control of a tissue specific promoter and a strong repressor which is regulated by drug. The cytotoxin is extremely toxic, such that even a small leakage of the tissue specific promoter outside of the tumor cells could potentially be toxic to the virus packaging cell. In turn, it is usually very difficult to generate high titer virus encoding cytotoxin protein. In certain embodiments, a tetracycline response element (TRE) is put upstream of the tissue specific promoter. The same construct will also express tetracycline-controlled transcriptional silencer (tTS). tTS protein binds tightly to the tetO sequences within the TRE and actively silences transcription of the toxin mRNA from the tissue specific promoter in the absence of Dox. In this basal state, background expression of the toxin gene is extremely low and will facilitate the production of virus. When the inducer drug (Dox) is added, tTS binds to Dox and dissociates from the TRE, relieving transcriptional suppression and permitting the toxin gene to be transcribed from the tissue specific promoter.

In certain embodiments, the expression constructs and vectors disclosed herein may be delivered via neuronal stem cells (NSCs). Therefore, provided herein in certain embodiments are NSCs that comprise a vector as disclosed herein, and methods for delivery of an expression construct as disclosed herein to the site of metastasis or the origin point of metastasis using these NSCs. In such methods, the NSCs act as both a producer and delivery vehicle of a viral vector such as AAV. The tumor-tropic properties of NSCs have been described previously (Aboody 2000; Aboody 2006), and the use of NSCs as delivery vehicles for treating tumors in the central nervous system and solid tumors throughout the body have been validated (Kim 2006, Danks 2007, Sims 2008). NSCs offer a novel method of targeting therapeutics agents selectively to metastatic tumor sites irrespective of tumor size or location, thereby providing a means of delivery that achieves a higher therapeutic index with limited toxicity to normal tissue.

When delivered regionally or intravenously, NSCs will migrate to and through primary brain tumors. For example, NSCs can cross the blood-brain barrier and migrate to and infiltrate bulk tumors in vivo (Brown 2003). Previous studies have also established the therapeutic efficacy of intracranially-delivered NSCs expressing the prodrug activating enzyme cytosine deaminase with systemically administered 5-fluorocytosine in brain tumor models of glioma, medulloblastoma, and melanoma brain metastases (Aboody 2006, Kim 2006, Danks 2007), as well as intravascularly-delivered NSCs secreting rabbit carboxylesterase with systemically administered CPT-11 (Irinotecan) in animal models of disseminated neuroblastoma. Therefore, because of their ability to cross the blood-brain barrier and their ability to target tumors, the NSCs provided herein allows for efficient delivery of the presently disclosed expression construct to tumors.

In certain embodiments, methods are provided for treating a malignant melanoma, including a malignant melanoma that has metastasized, comprising administering to a subject in need thereof one or more of the expression constructs provided herein. In certain of these embodiments, the construct is delivered in a vector, such as for example an AAV vector, and in certain of these embodiments the vector is delivered in a cell, such as for example an NSC. Expression constructs, vectors, and cells may be delivered locally, regionally or systemically using any route known in the art, such as for example, intravenous, intratumoral, intraventricular, intranasal, intraocularly or intracranial injection.

To increase the chance for achieving successful gene therapy, an expression vector is ideally delivered to the entire tumor. Therefore, in certain embodiments, delivery of expression constructs, vectors and cells as described above may utilize convection-enhanced delivery. Convection-enhanced delivery is a form of high flow micro-infusion developed for the central nervous system to distribute an expression vector through large volumes of tissue (Chen, M. Y. et al. 1999; Chen, M. Y. et al. 2005; Hamilton, J. F. et al. 2001). In convection-enhanced delivery, a catheter is stereotactically implanted into the brain tumor. A pump then generates a pressure gradient at the tip of a catheter resulting in precise, widespread, homogenous particle distribution through the extracellular space.

Convective delivery can distribute macromolecules and nanoparticles such as viruses (Chen, M. Y. et al. 1999; Chen, M. Y. et al. 2005; Hamilton, J. F. et al. 2001). It has also been demonstrated that convection-enhanced delivery can distribute potentially therapeutic agents safely and reproducibly through large tumors in clinical and laboratory settings (Bankiewicz, K. S. et al. 2000; Lonser, R. R. et al. 2002; Lonser, R. R. et al. 2007). Metastatic brain melanomas are good candidates for convection. These tumors are generally spherical like the distribution arising from the catheter tip in convection-enhanced delivery. Additionally, metastatic brain tumors are surrounded by a capsule that should contain the infusate within the tumor, increasing efficacy and safety.

In certain embodiments, gene delivery of the delivery of expression constructs, vectors and cells as described above may be enhanced by ultrasound-facilitated transduction to improve viral transduction. Ultrasound has been shown, both in vivo and in vitro, to have the capacity to significantly increase viral transduction, likely sonoporating the cell membrane, which allows viral entry.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### EXAMPLES

### Example 1

### mRNA Expression Profiles of Tyrosinase in Different Human Tissue And Cell Lines

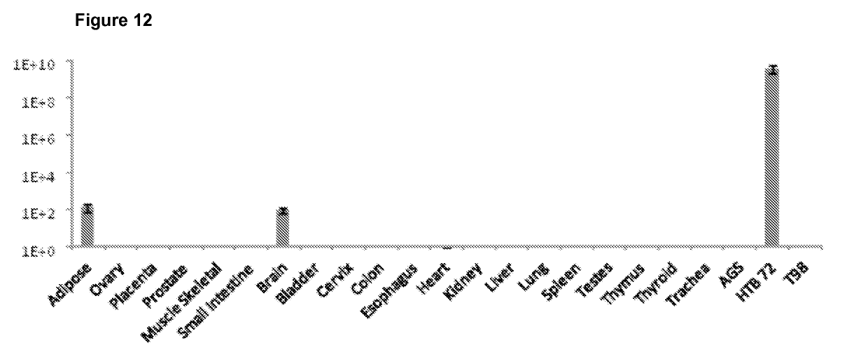

To confirm that tyrosinase is specifically expressed in melanocytes, quantitative measurements of tyrosinase mRNA levels were performed on a panel of 20 normal human tissues, a panel of four human melanoma cell lines, two human nonmelanoma cell lines and normal human cortex tissue. FIG. 12 shows the quantitative RT PCR results. The values are normalized with level of GAPDH. A trace amount of tyrosinase was detected in heart, and its value was set to 1. Tyrosinase was also detected in adipose and brain tissues. However, the expression level in brain was 10 million times less than in melanoma cells.

### Example 2

### Quantification of Tyrosinase mRNA Expression in Tumor and Non-Tumor Cell Lines

To confirm classification of melanoma and nonmelanoma cell lines, quantitative measurements of tyrosinase mRNA levels were performed on a panel of four human melanoma cell lines, two human nonmelanoma cell lines, and normal human cortex tissue. Tyrosinase mRNA levels were compared by quantitative real-time RT-PCR using WYC1 (melanoma) as a reference. Total RNA was isolated from cultured cells using a Qiagen RNA extraction kit (Qiagen), and cDNA was prepared from 2.0 μg RNA using 0.25 ng oligo-(dT)12-18 and reverse transcriptase according to manufacturer protocol.

Real-time PCR was performed using a Bio-Rad Sequence Detection System in the presence of SYBR-green. Beta-actin gene expression was used for normalization. Relative gene expression levels were presented as 2(-delta Ct) where delta Ct=Ct (target)-Ct beta-actin); Ct, cycle threshold. Primer sequences for beta-actin were 5′-ACAAAACCTAACTTGCGCAG-3′ (forward, SEQ ID NO:1) and 5′-TCCTGTAACAACGCATCTCA-3′ (reverse, SEQ ID NO:2), and primer sequences for tyrosinase were 5′-TCTTCTCCTCTTGGCAGATTGTC-3′ (forward, SEQ ID NO:3) and 5′-TGTCATGGTTTCCAGGATTACG-3′ (reverse, SEQ ID NO:4).

Results are summarized in Table 1. Tyrosinase mRNA expression level per unit RNA is reported as a percentage of the expression of WYC1. Expression of tyrosinase mRNA in the melanoma cell lines ranged from 100% to 550% of that in WYC1, whereas expression in nonmelanomas was almost undetectable. These results confirm that tyrosinase mRNA is selectively expressed in melanoma cells.

TABLE 1
Tyrosinase mRNA levels in melanoma and nonmelanoma cell lines

| | | Tyrosinase |
|---|---|---|
| | Cell line | mRNA level |
| Nonmelanoma | U251 | 0.01 |
| | A549 | 1.49 |
| | Normal human cortex | 0.1 |
| Melanoma | WYC1 | 100 |
| | A2058 | 280 |
| | HTB72 | 550 |
| | HTB65 | 480 |

### Example 3

### Assessment of In Vitro TyrP Activity in Tumor and Non-Tumor Cell Lines

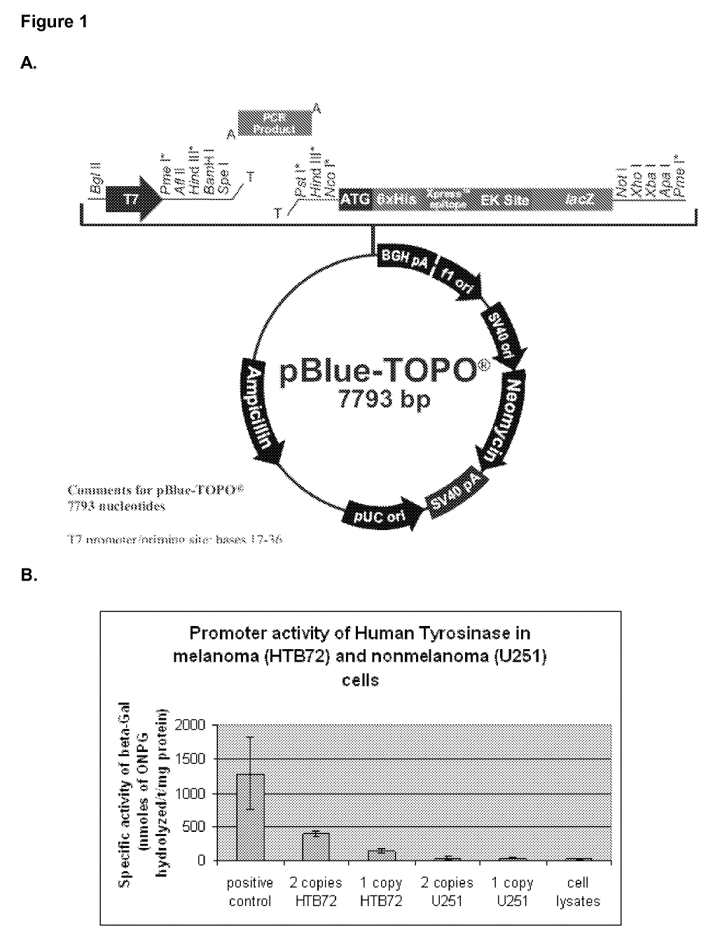

Native human TyrP (hTyrP) is located downstream of the enhancer element hE, which contains a tyrosine distal element (TDE) that enhances tissue-specific transcription of the tyrosinase gene. Previous studies have shown that at least two enhancers fused to the 260-bp core TyrP are required to obtain high and selective expression in melanoma cell lines in vitro (Shi 2002; Lillehammer 2005). Constructs containing hTyrP linked to one enhancer (hE-hTyrP) or two enhancers (2hE-hTyrP) were inserted into pBlue vector (Invitrogen, FIG. 1A), a promoter activity reporter, and transiently transfected into melanoma (HTB72) and nonmelanoma (U251) cells cultured in 10% fetal bovine serum/Dulbecco's modified Eagle's medium containing antibiotics and 2 mM Glutamine at 37° C. in a humidified atmosphere with 5% CO₂. Positive control cells were transfected with pBlue containing the beta-galactosidase reporter gene linked to the constitutively active CMV promoter. Untransfected cell lysates served as negative controls to determine background noise.

For the transfection, 1×10⁵ cells were seeded in a twenty four-well plate overnight, then transfected by the Lipofectamine 2000-mediated transfection method (Invitrogen). Ortho-nitrophenyl-D-galactopyranoside (ONPG) beta-galactosidase substrate was added to the cell lysates, and beta-galactoside activity was measured 48 hours after transfection by measuring absorbance at 420 nm. Statistical differences in promoter activity were analyzed by student 2-tailed t test, and results were considered significantly significant if P<0.05.

Tyrosinase promoter activity was almost undetectable in U251 nonmelanoma cells transfected with hE-hTyrP or 2hE-hTyrP, with beta-galactosidase levels similar to those seen in cell lysates (FIG. 1B). Melanoma cells transfected with the single enhancer construct exhibited significantly higher beta-galactosidase activity, and melanoma cells transfected with the double enhancer construct exhibited beta-galactosidase expression levels that were approximately 1,300% that of melanoma cell lysates (FIG. 1B). These results confirm that expression from an exogenous tyrosinase promoter is significant in melanoma cells but not nonmelanoma cells, and that linking the tyrosinase to two enhancers rather than one significantly increases expression.

### Example 4

### Assessment of Cell Killing by Saporin

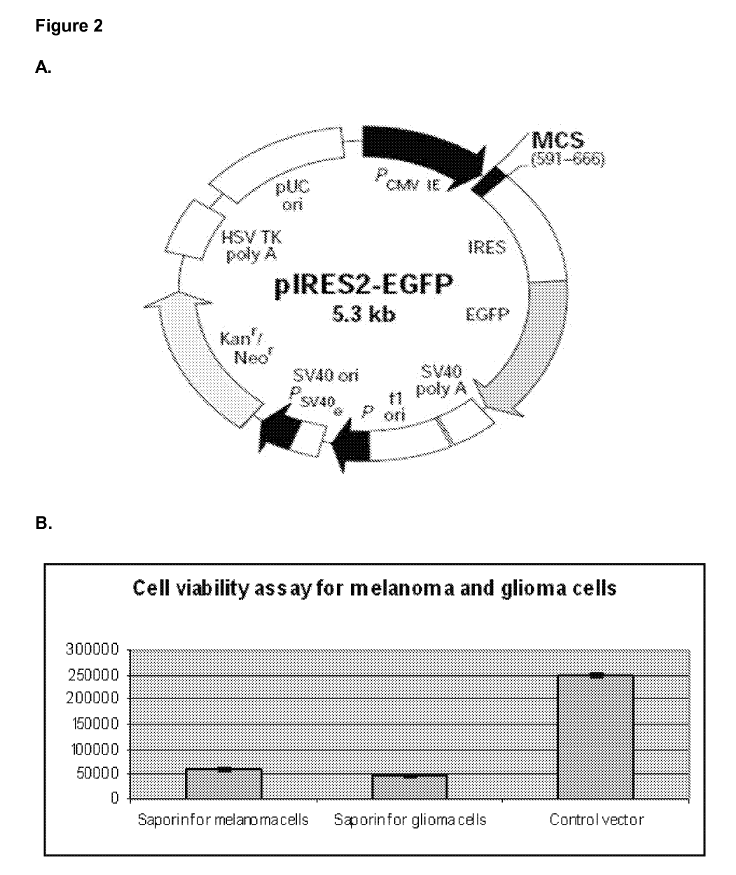

cDNA encoding a saporin gene having the sequence set forth in SEQ ID NO:5 was cloned into pIRES (Clontech, FIG. 2A), placing the gene under control of the CMV promoter. The vector was transiently transfected into melanoma and nonmelanoma cells using the techniques described above in Example 2, and a cell viability assay was performed after 48 hours using a CellTiter-Glo ATP assay (Promega). This assay generates a “glow” luminescent signal in the presence of ATP from viable cells, which is then detected using a plate reader luminometer.

The saporin gene was found to be cytotoxic to both melanoma and nonmelanoma cells, exhibiting a death rate of more than 75% for all cell types (FIG. 2B).

### Example 5

### Construction of TyrP-Saporin

pShuttle-2hE-hTyrP (Lillehammer 2005) contains the human minimal tyrosinase promoter (−209 to +51 by relative to the human tyrosinase transcription start site) and two enhancer elements (hE: −2014 to −1810 by relative to the human tyrosinase transcription start site). PCR amplification was carried out using the pShuttle-2hE-hTyrP plasmid as a template to generate hTyrP and one or two copies of hE. The first synthetic oligonucleotide utilized in the PCR reaction contained an AseI site and the first twenty codons of the 5′ enhancer site, while the second primer contained an NheI site and the twenty codons in the 3′ end of the hTyrP in the opposite orientation.

In each case, the amplified fragment was digested with AseI and NheI and ligated into AseI and NheI-digested pIRES2-EGFP vector (Clontech), resulting in replacement of the CMV promoter with TyrP. The resultant plasmids were used to subclone a cDNA fragment encoding saporin (Bagga 2003a; Bagga 2003b), resulting in construction of pIRES-2hE-hTyrP-saporin and pIRES-hE-TyrP-saporin. Sequence analysis was performed on the constructs to ensure that no sequence alterations occurred during amplification.

### Example 6

### Assessment of Cell Killing by TyrP-Saporin

Melanoma (HTB72 and WYC1) and nonmelanoma cells were transiently transfected with the TyrP-saporin constructs from Example 4 using the techniques described above in Example 1. A cell viability assay was performed after 48 hours using the CellTiter-Glo ATP assay.

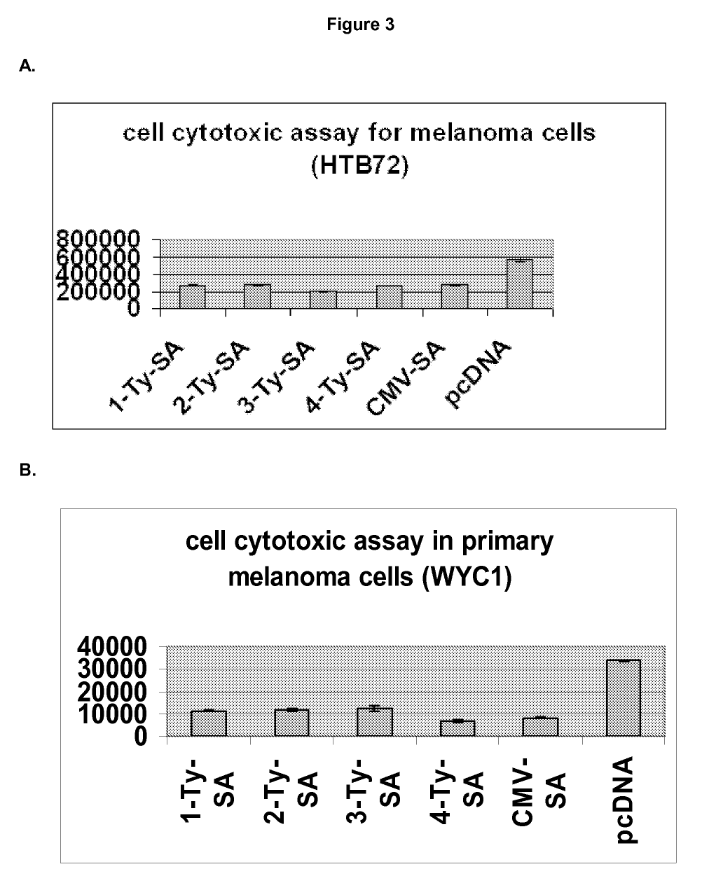

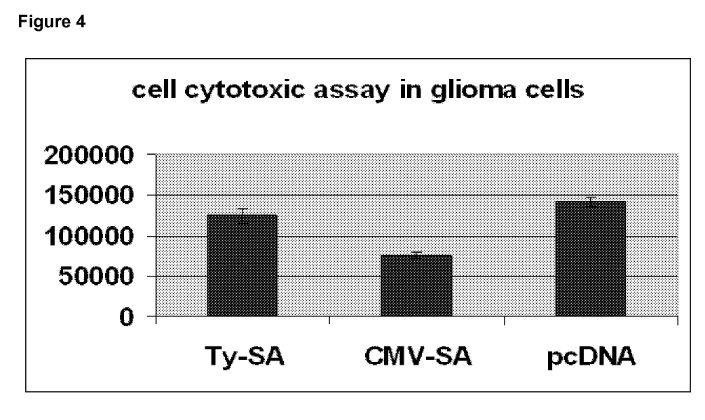

Transfection of nonmelanoma glioma cells with hTyrP-saporin constructs did not result in cytotoxicity (FIG. 4). Transfection of melanoma cells with either hTyrP-saporin construct, on the other hand, resulted in substantial cell killing (FIG. 3). Use of an hTyrP-saporin construct with three of four enhancer elements did not significantly increase cell cytotoxicity above that observed with two enhancer elements, indicating that expression of only a few saporin molecules is sufficiently toxic to melanoma cells. These results confirm that the TyrP-saporin constructs disclosed herein can be used to selectively kill melanoma cells while leaving nonmelanoma cells unharmed.

### Example 7

### Codon Optimization of Saporin-6 Gene

Sequence optimization was performed on the saporin-6 gene (SEQ ID NO:5) to optimize the gene for expression in humans. This optimization procedure was designed to avoid the following cis-acting sequence motifs where possible: 1) internal TATA boxes, chi-sites, and ribosomal entry sites, 2) AT-rich or GC-rich sequence stretches, 3) RNA instability motifs, 4) repeat sequences and RNA secondary structures, and 5) (cryptic) splice donor and acceptor sites. Regions of greater than 80% or less than 30% GC content were avoided when possible to prolong mRNA half-life. The optimized sequence (SEQ ID NO:6) had an average GC content of 59% over all possible 40 by stretches, and a Homo sapiens codon adaptation index (CAI) of 0.98. Based on this CAI, the optimized gene sequence should be highly and stably expressed in humans.

### Example 8

### Construction of a AAV Vector Expressing Saporin Under the Control of TyrP Promoter and tTS Silencer

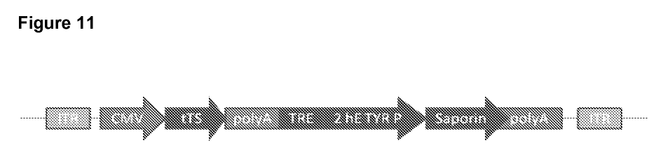

A parental AAV vector (pAAV MCS) will be engineered to encode the tetracycline-controlled transcriptional silencer (tTS), and a modified tetracycline response element will be put in the upstream of the multiple cloning sites. On the basis of this construct, the saporin gene under the control of tyrosinase promoter (TyrP-saporin) will be subcloned into the vector. The construct is shown in FIG. 11.

The construct will be transfected into the packaging cell to check if there is any promoter leakage, and will also be transfected into a melanoma cell (HTB65, HTB72 etc) to check the inducibility of the transcription of the toxin gene by Dox. This construct will be used to generate AAV, and high titer generation of the target virus is expected.

### Example 9

### Cytotoxicity of Optimized Saporin Linked to CMV Versus TyrP Promoters

A cDNA sequence comprising the optimized saporin-6 gene of Example 7 (SEQ ID NO:6) was subcloned into pIRES2-CMV-EGFP or pIRES2-2hE-TyrP-EGFP using the methods described above in Examples 4 and 5. The resultant vectors were transiently transfected into U251 glioma cells and HTB72 melanoma cells at concentrations of 0.7, 1.4, or 2.1 μg/well as described above in Example 3, and a cell viability assay was performed after 48, 72, or 96 hours using a CellTiter-Glo ATP assay (Promega).

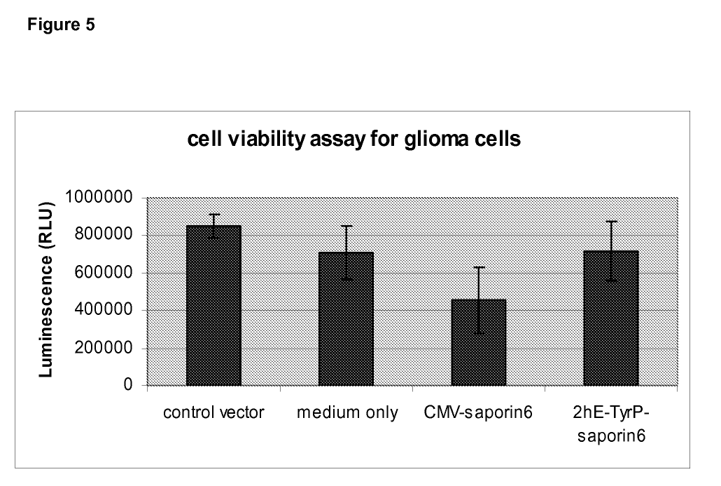

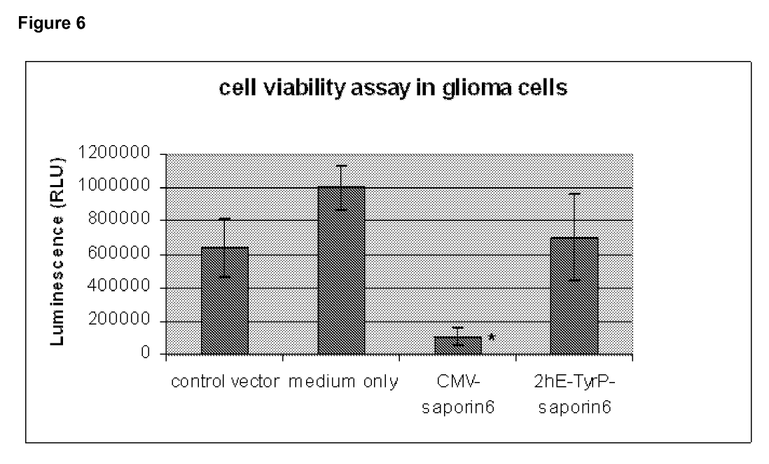

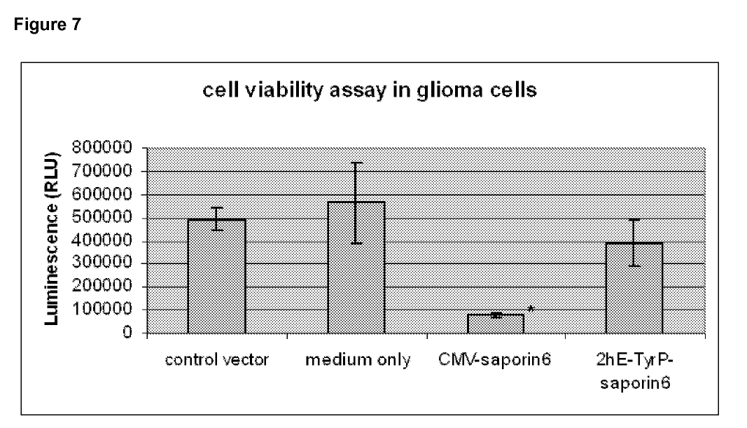

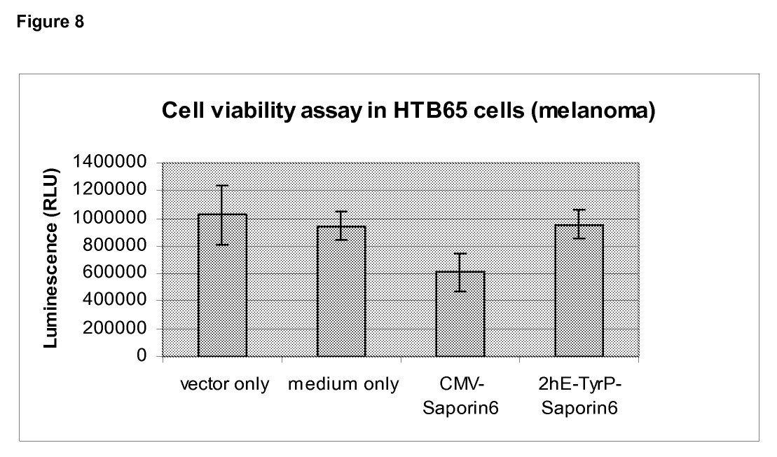

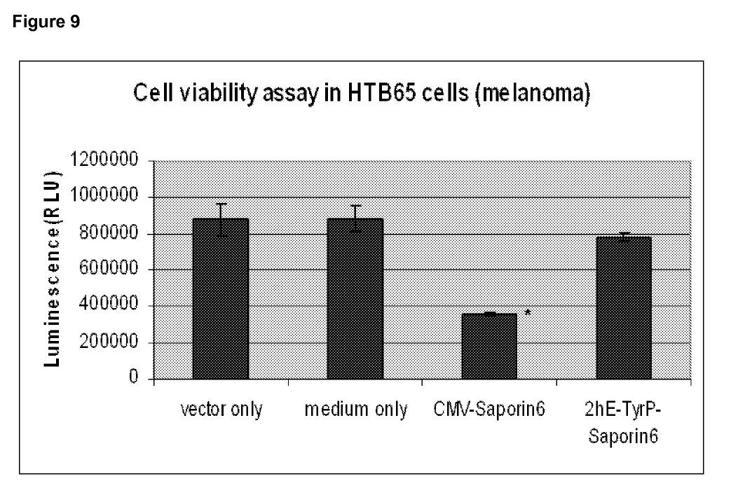

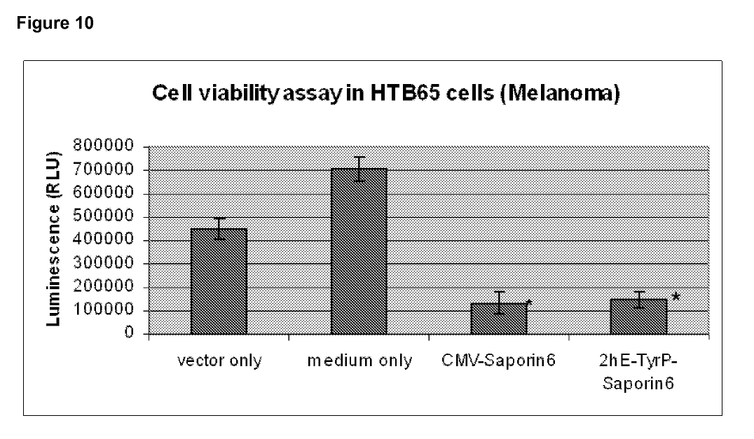

Significant cytotoxicity was observed in glioma and melanoma cells transfected with CMV-saporin vector (representative results shown in FIGS. 5-7 (glioma) and 8-10 (melanoma)). Significant cytotoxicity was also observed in U251 melanoma cells transfected with the highest concentration of 2hE-TyrP-saporin (FIGS. 8-10). Glioma cells transfected with 2hE-TyrP-saporin, on the other hand, exhibited cytotoxicity levels similar to control cells transfected with empty vector regardless of vector concentration (FIGS. 5-7). This confirms that TyrP-saporin constructs utilizing an optimized saporin codon sequence can be used to selectively kill melanoma cells while leaving nonmelanoma cells unharmed.

### Example 10

### Construction of an NSC that Produces AAV-TyrP-Saporin

An NSC will be constructed to produce AAV-TyrP-saporin, AAV-hE-TyrP-saporin, and/or AAV-2hE-TyrP-saporin using techniques known in the art. The saporin gene used in these constructs may be either the original saporin-6 gene (SEQ ID NO:5) or the optimized codon version described in Example 7 (SEQ ID NO:6). Control NSCs will be constructed to produce AAV-CMV-EGFP. AAV vector production will utilize a helper-free method based on adenovirus-free transient transfection of all elements required for AAV production in host cells such as HEK293 in which the E1A gene is expressed. The resultant NSCs will not only harbor integrated and rescuable vector plasmid DNA and AAV Rep and Cap genes, but will also contain the essential adenovirus helper genes.

Five adenovirus genes (E1A, E1B, E2A, E4, and VA RNA) are generally required for efficient AAV expression, DNA replication, and packaging. E1A not only positively controls the expression of other helper genes but also trans activates AAV Rep and Cap genes. Therefore, leaky expression of E1A will turn on adenovirus genes and the AAV Rep gene. The latter is well known to be cytotoxic and to induce accumulation of cells in the G1 phase of the cell cycle.

Due to the cytotoxicity associated with Rep, it is difficult to obtain a stable cell line from cells that constitutively express E1A. Therefore, Rep expression and/or activity may be reduced using a variety of techniques known in the art in order to generate a more stable cell line. In certain embodiments, Rep expression and/or activity may be reduced by mutating the Rep gene or a promoter associated with the Rep gene such as the p5 promoter. These mutations may be introduced in a targeted or random manner. In certain embodiments, the mutations result in decreased Rep expression, while in other embodiments Rep expression remains essentially unchanged but Rep activity is decreased, altered, or eliminated.

Mutations, deletions, and/or insertions may be introduced into the Rep gene using any of a variety of techniques known in the art. For example, mutations may be introduced through random mutagenesis using error-prone PCR, radiation, or chemical agents, targeted mutation using site-directed mutagenesis or homologous recombination, or insertion of one or more nucleotides or complete stop codons in a random or targeted manner. Previous studies have shown that mutations to Y224 of Rep78, which is involved in DNA binding and ATPase-helicase activities, reduces Rep toxicity. Therefore, in certain embodiments, targeted mutations may be employed that specifically target the codon encoding Y224.

In other embodiments, cytotoxicity associated with Rep may be reduced by completely or partially removing the Rep gene and/or a promoter associated with the Rep gene such as the p5 promoter, resulting in decreased Rep expression. In certain of these embodiments wherein the Rep gene is completely or partially removed, the Rep gene may be replaced with one or more functionally equivalent genes with reduced cytotoxicity. In certain of these embodiments wherein the promoter sequence is completely or partially removed, the promoter may be replaced with a minimal promoter that decreases Rep expression. The replacement of the Rep p5 promoter has been used previously to generate adenoviruses that express only low levels of Rep and therefore produce high titers of AAV vectors. In still other embodiments, cytotoxicity associated with Rep may be decreased at the protein level, for example using polypeptides that specifically bind to and inhibit Rep.

Removal of sequences coding for Rep from AAV vectors has been shown to decrease the ability of AAV to preferentially integrate into the AAVS1 site on human chromosome 19. A variety of techniques have been developed for overcoming this limitation when the Rep gene is removed from the AAV vector or otherwise disabled. For example, cells may be transfected with the Rep protein directly, or Rep expression may be regulated using a Cre-loxP recombination-based system. Alternatively, the Rep gene may be fused to a hormone-dependent ligand-binding domain such that it can only be transported into the nucleus in the presence of a hormone analogue. In another approach, Rep mRNA transfection is utilized to facilitate transient expression of Rep68/78 protein. mRNA only has to reach the cytoplasm to be expressed, thereby circumventing the process of transport into the nucleus. Using these and other techniques, the benefits of Rep expression may be maintained while preventing or reducing the cytotoxicity associated with long-term or high level expression of Rep.

Certain constructions of NSCs will utilize HB1.F3, a well-characterized, v-myc-immortalized, nontumorigenic clonal cell line derived from human fetal telencephalon. HB1.F3 will initially be modified so that tTA is constitutively expressed. tTA is a fusion product of the amino terminal-DNA binding domain of the tet repressor and the carboxy-terminal activation domain of VP-16 from herpes simplex virus. In the absence of tetracycline, tTA binds to the tet-responsive elements (TRE) in the tet operator and efficiently activates transcription from downstream minimal promoters. The association between tTA and the TRE is prevented by tetracycline; therefore, in the presence of low concentrations of tetracycline or its derivative deoxycycline, transcription from TRE is turned off.

To generate inducible E1A-E1B cell lines, the tetoff system will be used to regulate E1A gene expression. In the presence of TET or its analog DOX, the E1A gene should be repressed, while the removal of TET or DOX should turn on the E1A gene, subsequently activating the E1B gene. The inducible pST-E1AB plasmid will be cotransfected with a puromycin resistance plasmid into the tTA HB1.F3 cell line. The cell line expresses a TET repressor-VP16 fusion protein that activates the TRE promoter, whereas the presence of DOX abolishes the activation and represses the TRE promoter. After selection with puromycin, NSC AAV producer cell lines for production will be obtained. To examine whether those cells can express the E1A and E1B genes upon removal of DOX, ELISA or Western blot will be used. For subsequent experiments, we will choose the clone with the highest expression.

Generation of AAV-CMV-EGFP, AAV-TyrP-saporin, AAV-hE-TyrP-saporin, and AAV-2hE-TyrP-saporin from HB1.F3 NSCs will require the use of three plasmids: one carrying the transgene with AAV ITRs, one carrying the AAV replication (rep) and capsid (cap) genes, and one carrying the adenovirus helper genes E2, E4, and VA RNA genes. The cytotoxin or EGFP gene will be cloned into multiple cloning sites of rAAV2 ITRs plasmid, and the CMV promoter will be replaced by TyrP, hE-TyrP, or 2hE-TyrP. The crucial role of ITRs in the AAV life cycle occurs during transfection of the rAAV plasmids into tTA HB1.F3 which can provide E1A protein once DOX is removed from medium resulting in successful rescue, replication and packaging of infectious mature virions by co-transfection with AAV Rep, Cap and helper genes from a non-rescuable plasmid. Initial methods of rAAV2 production will involve cotransfection of these plasmids into 150 mm dishes of NSC producer cells. The next day, DOX will be removed, and approximately 72 hours after the transfection rAAV2 will be collected from cell culture supernatant and the physical and infectious titers of rAAV preparations will be determined by titer assay. The NSC inducible cell line is expected to possess the same ability as HEK293 cells to produce helper-free, high titer AAV.

### Example 11

### Assessment of Cell Killing by NSCs Producing AAV-TyrP-Saporin

The cytotoxicity of the NSCs generated in Example 10 will be tested in vitro in various melanoma (e.g., WYC1, HTB65, HTB72, B16) and nonmelanoma (U251, T98, MEF) cell lines using methods similar to those described above in Examples 4 and 6. Based on the results disclosed above, it is expected that NSCs containing TyrP-saporin will be cytotoxic to melanoma cells but non-cytotoxic or minimally cytotoxic to nonmelanoma cells.

The effect of increasing dosages of NSC-AAV-TyrP-saporin will be assessed by labeling melanoma and nonmelanoma cells with yellow fluorescent protein (YFP), incubating these cells with NSCs, and measuring cytotoxicity based on loss of YFP signal at days 3-7. It is expected that administration of NSCs containing AAV-TyrP-saporin will deplete the population of melanoma cells in a dose- and time-dependent fashion.

### Example 12

### Convection-Enhanced Delivery of Cy3-Labeled AAV-hE-TyrP-GFP

The ability of convective-enhanced delivery to effect widespread, specific distribution throughout a tumor can be tested in vivo with a murine model. C57 mice are implanted intracranially with B16 luciferase-expressing melanoma cells as previously described (Craft, N. et al. 2005). Starting one week after implantation, animals may be evaluated for tumor size, such as by injecting with luciferin substrate and imaging using the Xenogen IVIS bioluminescent imager.

When tumor size reaches >3 mm in diameter, Cy3-labeled AAV-hE-TyrP-GFP (at approximately 1×10¹² viral particles/ml) can be stereotactically convected into tumors as previously described (Chen, M. Y. et al. 2005). Briefly, anesthetized animals are affixed in a Kopf stereotactic frame. A cannula is laced through a cranial burr hole into the center of the tumor. An infusion pump is then attached to the cannula to generate convective flow at a rate of 0.1 μl/minute. The volume of infusion necessary to “fill” a tumor is typically ⅕ of the volume of the tumor. The infusion rate may be optimized and the volume of infusion to volume distribution ratio, which is typically a linear relationship, may be determined as previously described (Chen, M. Y. et al. 1999).

Animals should be imaged at several timepoints: prior to tumor implantation, immediately before and after virus delivery, and three and seven days after vector delivery. The Xenogen IVIS bioluminescent imager will be used to determine distribution of Cy3 capsids and GFP expression relative to the size and location of the B16 luciferase-expressing tumors. The use of different filters will allow independent quantification of Cy3, GFP and luciferin. Immediately after convection, the distribution of Cy3 (capsids) should match that of luciferin (tumor), indicating that efficient distribution of the vectors throughout the tumor. Three to seven days after viral injection, the GFP signal (transgene expression) should match that of luciferin as well, indicating efficient and complete viral transduction.

### Example 13

### Assessment of Cytotoxicity for Convectively Delivered Cy3-Labeled AAV-hE-TyrP-Saporin to Murine Metastatic Brain Melanoma

The cytotoxicity of convective-enhanced targeted delivery of AAV-hE-TyrP-Saporin in murine metastatic brain melanoma may be tested in vivo using B16 luciferase-expressing melanoma tumors in C57 mice as described in Example 12. Briefly, AAV-hE-TyrP-Saporin, AAV-TyrP-eGFP (vector control) and normal saline (vehicle) may be convectively delivered into intracranial B16 luciferase-expressing melanoma tumors in C57 mice. Animals will be bioluminescence-imaged immediately before and after viral delivery to insure tumor size (by luciferin) of at least 3 mm and adequate distribution of viral capsids (by Cy3). Initially, the IC50 dose of AAV-hE-TyrP-Saporin will be used, which is determined in vitro. Animals will be monitored for tumor size, neurological status and survival 3, 7, 14 and 28 days after treatment. On day 28, or earlier if the animal does not survive, the mice will be euthanized for brain sectioning and microscopic verification of tumor size. Viral doses will then be adjusted with the goal of complete tumor eradication. Treatment with AAV-hE-TyrP-Saporin should cause tumor necrosis, reduced tumor size, and prolonged survival, and slight overflow into normal adjacent brain should cause minimal injury.

The effect of the therapeutic vector convected directly into normal murine brain should be a good measure of safety. The toxic effects of AAV-hE-TyrP-Saporin, AAV-CMV-Saporin (positive control) and AAV-hE-Tyr-eGFP (vector control) will be examined as well. Following convection of virus, the animals' general health and neurological status will be monitored for four weeks after which they will be euthanized. Brain sections will be analyzed for markers of inflammation, cellular infiltration, and tissue damage. Fixed brains will be sliced into 40 μm sections. One series (every six section) will be stained with anti-DARPP-32, a sensitive cytoplasmic indicator of striatal tissue damage, and then counterstained with hematoxylin to reveal tissue morphology and infiltration of leukocytes into the brain parenchyma. In addition, sections will be stained for the following markers of innate immunity and neuroinflammation: GFAP, which is normally expressed by astrocytes in response to damage, and OX-42, a marker that is upregulated in activated microglial cells and macrophages.

As stated above, the foregoing are merely intended to illustrate the various embodiments of the present invention. As such, the specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference in their entirety as if fully set forth herein.

### REFERENCES

The references listed below and all references cited above are hereby incorporated by reference in their entirety, as if fully set forth herein.
1. Aboody et al. 2000. Proc Natl Acad Sci USA 97:12846.
2. Aboody et al. 2006. Neuro Oncol 8:119.
3. Bagga et al. 2003a. FEBS Lett 541:16.
4. Bagga et al. 2003b. J Biol Chem 278:4813.
5. Benatti et al. 1989. Eur J Biochem 183:465.
6. Benatti et al. 1991. FEBS Lett 291:285.
7. Bankiewicz et al. 2000. Exp. Neurol. 164:2-14.
8. Brown et al. 2003. Hum Gene Ther 14:1777.
9. Chen et al. 1999. J. Neurosurg. 90:315-320
10. Chen et al. 2005. J. Neurosurg. 103:311-319.
11. Craft et al. 2005. J. Invest. Dermatol. 125:159-165.
12. Danks et al. 2007. Cancer Res 67:22.
13. Hacker et al. 2005. J Gene Med 7:1429.
14. Hamilton et al. 2001. Exp Neurol 168:155-161.
15. Kim et al. 2006. Clin Cancer Res 12:5550.
16. Lillehammer et al. 2005. Cancer Gene Ther 12:864.
17. Liu et al. 2007. Cancer Treatment Rev 33:665.
18. Lonser et al. 2002. J. Neurosurg 97:905-913.
19. Lonser et al. 2007. Neurology 68:254-261.
20. Shi et al. 2002. Hum Gene Ther 13:211.
21. Siders et al. 1996. Cancer Res 56:5638.
22. Sikriwal et al. 2008. Int J Biochem Cell Biol 40:2880.
23. Sims et al. 2008. Ann Surg Oncol 15:3259.
24. Stirpe et al. 1992. Biotechnology 10:405.
25. Stirpe & Battelli. 2006. Cell Mol Life Sci 63:1850.
26. Toyoda et al. 2004. Gene 332:61.
27. Zarovni et al. 2007. Cancer Gene Ther 14:165.

1. An expression construct comprising a tissue-specific promoter operably linked to one or more cytotoxin genes.

2. The expression construct of claim 1, further comprising one or more enhancer elements.

3. The expression construct of claim 1, wherein said tissue-specific promoter is a tyrosinase promoter.

4. The expression construct of claim 1, wherein said one or more cytotoxin genes comprise one or more genes encoding ribonucleotide inactivating proteins.

5. The expression construct of claim 4, wherein said one or more ribonucleotide inactivating proteins comprise saporin.

6. The expression construct of claim 5, wherein said one or more genes encoding saporin comprise the nucleotide sequence set forth in SEQ ID NO:5.

7. The expression construct of claim 5, wherein said one or more genes encoding saporin comprise the nucleotide sequence set forth in SEQ ID NO:7.

8. A vector comprising the expression construct of claim 1.

9. The vector of claim 8, wherein said vector is selected from the group consisting of an animal virus vector, a hybrid animal virus vector, and a liposome.

10. The vector of claim 9, wherein said animal virus vector is selected from the group consisting of adeno-associated virus and adenovirus.

11. A neural stem cell comprising the vector of claim 8.

12. A method of treating melanoma in a subject in need thereof comprising delivering a therapeutically effective amount of an expression construct of claim 1.

13. The method of claim 12, wherein said expression construct is delivered via a vector of claim 8.

14. The method of claim 13, wherein said vector is delivered via a neural stem cell of claim 11.

15. The method of claim 12, wherein said melanoma is malignant.

16. The method of claim 15, wherein said melanoma has metastasized.

17. The method of claim 14, wherein the neural stem cell is delivered by intravenous, intratumoral, intraventricular, intranasal, intraocularly or intracranial injection.

18. The method of claim 13, wherein said vector is delivered by convection-enhanced and/or ultrasonic delivery.
